(19) United States
(12) Patent Application Publication
(10) Pub. No.: US 2010/0310616 A1
(43) Pub. Date: Dec. 9, 2010

(54) METHOD OF COSMETIC CARE STIMULATING MITOCHONDRIAL ACONITASE

(75) Inventors: Carine Nizard, Ivry Sur Seine (FR); Marielle Moreau, Marcq (FR); Jean-Christophe Archambault, Meung Sur Loire (FR); Bertrand Friguet, Paris (FR); Anne-Laure Bulteau, Paris (FR)

Correspondence Address: MERCHANT & GOULD PC, P.O. BOX 2903, MINNEAPOLIS, MN, 55402-0903, US

(73) Assignee: LVMH RECHERCHE, Saint Jean de Braye (FR)

(21) Appl. No.: 12/751,677

(22) Filed: Mar. 31, 2010

(30) Foreign Application Priority Data
Mar. 31, 2009 (FR) 0952050

(51) Int. Cl. A61K 8/02; A61K 8/97; A61Q 19/08; A61K 8/49

(52) U.S. Cl. 424/401; 424/736; 424/725; 514/274

(57) ABSTRACT

The invention relates to a cosmetic care method for preventing or retarding the appearance of the signs of ageing of the skin or for attenuating the effects thereof.

This method is characterized in that it comprises the delivery, to at least part of the skin of the face or body, of an effective amount of at least one cosmetically acceptable active agent that activates or stimulates the activity of cutaneous mitochondrial aconitase.

The invention makes it possible to achieve an anti-ageing effect.

This application claims the benefit of Serial No. 0952050, filed Mar. 31, 2009 in France and which application is incorporated herein by reference. A claim of priority to all, to the extent appropriate is made.

The present invention relates to an anti-ageing cosmetic care method by stimulating the activity of mitochondrial aconitase.

More particularly, the invention relates to molecules or extracts, in particular of plant origin, which stimulate the activity of mitochondrial aconitase in the skin, to their use as active agents in cosmetic compositions, and to cosmetic care methods using the said compositions.

### TECHNICAL BACKGROUND

Ageing is a multi-factor phenomenon. Several theories exist regarding ageing, among which is the free radical theory based on the chemical nature and ubiquitous presence of these radicals (Harman D., J. Gerontol., 1956; 11, 298-300).

These free radicals, also known as reactive oxygen species (ROS), may be of exogenous origin or produced via various cellular processes, especially during mitochondrial respiration (Cadenas E. et al., Free Radic. Biol. Med., 2000; 29, 222-230).

During respiration, a small but significant amount of the total oxygen consumed by the respiratory chain is converted into superoxide radical O₂²⁻, which may lead to the formation of other even more reactive oxygen species, for instance hydrogen peroxide H₂O₂, and hydroxyl and peroxynitrite radicals (cf. Cadenas et al., cited above).

Under conditions of oxidative stress, the formation of these reactive species results in oxidative damage to proteins, DNA and lipids, and also to changes in the expression of the mitochondrial proteins and contributes towards the process of ageing of the skin (Bulteau et al., Exp Gerontol, 2006, 41; 653-657).

During ageing, reduced efficacy of the system for maintenance of cellular macromolecules and a constant increase in the production of ROSs in the mitochondria are observed (Humphries et al., Free Radic Res, 2006; 40, 1239-1243).

Now, the mitochondrion plays an important role in many cell functions, including the production of the proton gradient established by the respiratory chain and the production of ATP via the Krebs cycle (Liu et al., J. Neurochem., 2002; 80, 780-787).

Accumulation of oxidative damage and structural anomalies brings about in the mitochondrion the gradual loss of its capacity to produce the ATP required for the functioning and integrity of the cell (Frenzel et al., 1984).

Aconitase is an essential mitochondrial enzyme of the Krebs cycle, which converts citrate into isocitrate. It also plays a role in the preservation of mitochondrial DNA. By means of aconitase, the stability and hereditary transmission of this DNA are thus closely linked to the metabolic state of the cell (Chen et al., Proc Natl Acad Sci USA, 2007; 104, 13738-13743).

The activity of aconitase depends on the integrity of its iron-sulfur centre [4Fe-4S]²⁺ (Beiner, et al., Faseb J, 1993; 7, 1442-1449). Attack of its iron-sulfur centre [4Fe-4S]²⁺ by oxidizing agents brings about the formation of an iron-sulfur centre [3Fe-4S]⁺ which inactivates the aconitase.

The loss of activity of mitochondrial aconitase is an intracellular indicator of oxidative damage and cell ageing. Many degenerative disorders are also associated with the increase in the levels of pro-oxidative agents and the drop in activity of aconitase in the mitochondrion (Bulteau et al., Biochemistry, 2003 42, 14846-14855). Inactivation of aconitase especially brings about a change in the NADH/NAD⁺ ratios. Specifically, the production of NADH by α-ketoglutarate dehydrogenase and isocitrate dehydrogenase will be lowered in the Krebs cycle due to the fall in activity of aconitase (cf. Humphries et al., cited above; Nulton-Persson et al., J Biol Chem, 2001, 276, 23357-23361). Under these conditions of NAD⁺ accumulation, an increase in ROSs is observed on account of the autoxidation of the reduced metabolites. Such an inactivation may also initiate a cascade of oxidative reactions that contributes towards the accumulation of damaged proteins (cf. Humphries et al., 2006, cited above). It is thus essential, during ageing, to maintain a sufficient degree of activity of mitochondrial aconitase in skin cells, in order to prevent the accumulation of such cell damage and to promote the processes of repair and reactivation of the aconitase damaged by these ROSs.

### SUMMARY OF THE INVENTION

The inventors of the present invention have demonstrated that the activity of mitochondrial aconitase decreases by about 85% in human dermal fibroblasts in culture obtained from donors 70 years old, in comparison with those obtained from donors 20 years old, without any change in expression of the protein with age. Starting from this finding, they demonstrated that it is possible to totally protect mitochondrial aconitase and to re-establish its activity in these aged fibroblasts in culture, by treating the said fibroblasts with molecules or extracts of plant origin, whereas this same treatment applied to the fibroblasts of a young donor does not modify the activity of the mitochondrial aconitase.

Such an effect on stimulating the activity of mitochondrial aconitase enables this enzyme to conserve its central role in the Krebs cycle and in the preservation of the mitochondrial DNA, and also to preserve the functioning, especially the metabolic functioning, of skin cells.

This results from re-establishment of the activity of the mitochondrial aconitase of the skin cells of elderly individuals, an effect of slowing down the ageing of skin cells being reflected by an anti-ageing cosmetic effect.

### AIMS OF THE INVENTION

A main aim of the invention is to provide molecules or extracts, in particular of plant origin, which stimulate the activity of cutaneous mitochondrial aconitase, their use as active agents in cosmetic compositions, and cosmetic care methods using the said compositions.

A main aim of the invention is also to provide a method of anti-ageing cosmetic care by stimulating the activity of cutaneous mitochondrial aconitase.

A main aim of the invention is to propose the use of cosmetically acceptable molecule(s) or plant extract, as active agent in cosmetic compositions.

An aim of the invention is also the use of the said molecule or the said extract as an active agent in anti-ageing cosmetic compositions, and cosmetic care methods using the said compositions for preventing or retarding the appearance of the signs of ageing of the skin, or for slowing down the effects thereof.

### DESCRIPTION OF THE INVENTION

One subject of the present invention is thus directed towards a cosmetic care method for preventing or retarding the appearance of the signs of ageing of the skin or for attenuating the effects thereof, the said method being characterized in that it comprises the delivery, to at least part of the skin of the face or body, of an effective amount of at least one cosmetically acceptable active agent that activates or stimulates the activity of cutaneous mitochondrial aconitase.

Another subject of the present invention is thus also directed towards a cosmetic care method for caring for damaged skin, especially skin damaged by ultraviolet radiation, the said method being characterized in that it comprises the delivery, to at least part of the skin of the face or body, of an effective amount of a cosmetically acceptable active agent that activates or stimulates the activity of cutaneous mitochondrial aconitase.

According to one particular characteristic of the methods of the invention, the topically delivered active agent is incorporated into a cosmetic composition also comprising at least one cosmetically acceptable excipient.

The acceptable cosmetic active agent that stimulates the activity of mitochondrial aconitase may be a purified molecule, of natural or synthetic origin, or alternatively may be the product of a process of extraction from a raw material, in particular a raw material of plant origin.

According to one particular embodiment of the invention, the abovementioned cosmetic active agent comprises, or is essentially formed from, a plant extract obtained from at least one plant chosen from the group comprising those belonging to the genus Citrus or hybrids obtained from the crossing of plant species, at least one of which belongs to the genus Citrus, those belonging to the genus Morinda or those belonging to the genus Hibiscus.

According to another particular embodiment of the invention, the abovementioned active agent comprises, or is essentially formed from, a plant extract obtained from at least one hybrid belonging to the genus×Citrofortunella, resulting from the crossing of the genera Citrus and Fortunella, and in particular Calamondin (×Citrofortunella microcarpa).

According to one particular embodiment, the abovementioned active agent is an extract of Calamondin fruit.

According to another particular embodiment of the invention, the abovementioned active agent comprises, or is essentially formed from, a plant extract obtained from at least one plant belonging to the plant species Morinda citrifolia.

According to one particular embodiment variant, the abovementioned active agent is an extract of Morinda citrifolia fruit.

According to yet another particular embodiment of the invention, the abovementioned active agent comprises, or is essentially formed from, a plant extract obtained from at least one plant belonging to the plant species Hibiscus sabdariffa.

According to one particular embodiment variant, the abovementioned active agent is an extract of Hibiscus sabdariffa blossom.

The plant material used for the preparation of the extract may be the whole plant or a part of the plant such as the root, the rhizome or an aerial part, especially the stem, the leaves, the flowers, the seeds, the fruit or the floral buds.

It may advantageously be formed from the whole fruit or a part of the fruit of one of the species mentioned above.

A preferred extract is obtained from the flowers of the plant species Hibiscus sabdariffa. Such an extract is sold, for example, under the name Acides de Fleurs® by the company Greentech.

A preferred extract is obtained from calamondin fruit.

Another preferred extract is obtained from the fruit of Morinda citrifolia. Such an extract is sold, for example, under the name Citrifoline® by the company Solabia.

Before the extraction step per se, the plant material may have been dried and/or ground, or alternatively may be in the freshly harvested state.

The extract may be prepared via various extraction processes known to those skilled in the art.

The extraction may be performed without solvent, for example by pressing, especially of a whole fruit or of part of a fruit.

However, the extraction is advantageously performed by placing the selected plant material in contact with a polar solvent or a mixture of polar solvents, especially by soaking, maceration or decoction of the said plant material in the appropriate solvent or solvent mixture.

The polar solvent is advantageously chosen from water, a C1-C4 alcohol, in particular ethanol or butanol, a glycol preferentially chosen from glycerol, butylene glycol and propylene glycol, and mixtures thereof.

The preferred mixtures are mixtures of at least one alcohol and water or of at least one glycol and water, comprising at least 10% v/v of alcohol or glycol, the remainder being constituted by water.

The extraction may also optionally comprise an additional step that consists of a treatment of the plant material or plant extract, aimed at partially or totally decolorizing it or purifying it, for example via a treatment of the plant material or the extract with a solution of an apolar solvent or solvent mixture or via a treatment that consists in placing the extract in contact with active charcoal particles, or alternatively via a treatment with supercritical CO₂.

The extraction may be completed by a step of partial or total removal of the extraction solvents. In the first case, the extract is generally concentrated until an aqueous concentrate freed of significant amounts of organic solvent is obtained, and in the second case, a dry residue is obtained. Alternatively, the product from the extraction step may be freeze-dried or atomized in the form of a powder.

The powder may be used in the form as obtained, in a cosmetic composition according to the invention, or may be dispersed or dissolved in a polar solvent or a mixture of polar solvents, or alternatively may be adsorbed onto a solid support.

According to one embodiment variant of the present invention, the agent for stimulating the activity of mitochondrial aconitase is delivered topically in the form of a cosmetic composition comprising the said agent and at least one cosmetically acceptable excipient, by applying this composition to the skin of the body or the face, or to the integuments.

The cosmetic composition according to the invention comprises an amount of extract of the invention that is effective to produce the desired effect.

For any aspect of the invention, the term “effective amount” means an amount that is at least equal to the amount needed to prevent or retard the appearance of the signs of ageing of the skin or to attenuate the effects thereof.

The cosmetic composition according to the invention advantageously comprises from 0.001% to 5% by weight and preferably between 0.01% and 3% by weight of the composition, as active agent that stimulates the activity of mitochondrial aconitase.

The composition may also advantageously comprise other active agents that have cosmetic effects similar and/or complementary to those of the invention, and at least one cosmetically acceptable excipient that may be chosen especially from pigments, dyes, polymers, surfactants, rheological agents, fragrances, electrolytes, pH modifiers, antioxidants and preserving agents, and mixtures thereof.

The cosmetic composition according to the invention may be formulated, for example, in the form of a serum, a lotion, a cream, a hydrogel, preferably a mask, or may be in the form of a stick or a patch.

Finally, the present invention relates to a use of the active agents as defined above as cosmetic active agents for preventing or retarding the appearance of the signs of ageing of the skin or for treating them, the said cosmetic agent stimulating the activity of the mitochondrial aconitase of skin cells.

The invention also relates to a use of the cosmetic active agent of the invention for the manufacture of a cosmetic composition for preventing or retarding the appearance of the signs of ageing of the skin or for treating them.

Other aims, characteristics and advantages of the invention will emerge clearly from the explanatory description that follows, which is given with reference to several examples of implementation of the invention that are given purely for illustrative purposes and shall not in any way constitute a limitation of the scope of the invention.

In the description that follows and in the examples, all the percentages are given on a weight basis, the temperature is in degrees Celsius, and the pressure is atmospheric pressure, unless otherwise indicated.

### DESCRIPTION OF THE FIGURES

FIG. 1 relates to the modulation of mitochondrial aconitase activity with age, measured after culturing and treating human fibroblasts obtained from donors 20 and 70 years old, according to Example 2: (A) measurement of the mitochondrial aconitase activity (cf. Ex. 2, paragraph 3); (B) mitochondrial aconitase assay by Western blotting (WB) (cf. Ex. 2, paragraph 4).

FIG. 2 relates to modifications of the active site of mitochondrial aconitase with age, measured after culturing and treating human fibroblasts obtained from donors 20 and 70 years old, according to Example 2; separation and measurement of the forms of mitochondrial aconitase by immunoelectrofocusing (IEF) and then Western blotting (Ex. 2, paragraph 5).

FIG. 3 relates to the measurement of the mitochondrial aconitase activity, measured after culturing human fibroblasts obtained from donors 20 and 70 years old, cultured according to Example 2, and then treated with cosmetic active agents for 48 hours (cf. Ex. 2, paragraph 3): extract of Hibiscus sabdariffa 1.5%; extract of calamondin 2.5%; extract of Morinda citnfolia 2%.

### EXAMPLES

### Example 1

### Preparation of a Calamondin Extract Used for Performing the Tests

The extract of calamondin (×Citrofortunella microcarpa) is prepared by pressing whole fruit of the plant.

After filtering and then centrifuging, the fruit pulp is extracted with a mixture of water and polyglycerol.

The extract obtained contains 35-45% by weight of solids and is used for the tests of Example 2, and also for the manufacture of cosmetic compositions, especially that of Example 3.

### Example 2

### Measurement of Mitochondrial Aconitase Activity in Human Fibroblasts

Materials and Methods

1. Culturing of the Human Fibroblasts and Treatment

Extracts Tested
calamondin extract according to Example 1
aqueous extract of Hibiscus sabdariffa blossom, sold under the name Acides de Fleurs® by the company Greentech, comprising 22-25% by weight of solids,
water-glycol extract of Morinda citrifolia leaves, sold under the name Citrifoline® by the company Solabia, comprising 1.5% to 1.8% by weight of solids.

Media and Reagents

Fibroblast Culture Medium
DMEM 1/ml of glucose (Gibco)
+10% SVF
+1% sodium pyruvate 100 mM (Gibco)

Stock Solutions of Active Agents

The stock solutions are prepared in DMEM medium, from commercial solutions (% expressed as a percentage relative to the extract solution),
extract of Hibiscus sabdariffa at 6% by weight in the medium,
extract of Morinda citnfolia at 6% by weight in the medium,
calamondin extract at 6% by weight in the medium, i.e. about 2.4% by weight of solids.

a—Cell Culturing and Treatment

Human fibroblasts in primary culture obtained from plastic surgery of a donor 20 years old and of a donor 70 years old, at the 12th passage.

Subculturing on D0

Fibroblasts 15.10⁵ cells/dish of 75 cm² in triplicate in DMEM medium (10 ml/dish)

Treatment on D5

The stock solutions are diluted in the DMEM medium to produce the concentrations mentioned below:
extract of Hibiscus sabdariffa at 1.5% by weight in the medium
extract of Morinda citnfolia at 2% by weight in the medium
calamondin extract at 2.5% by weight in the medium, i.e. about 1% by weight of solids.

Recovery of the Cells on D7

Preparation of the Mitochondria

2 rinses with PBS

on a bed of ice; recovery in 2 ml of homogenization buffer.

b—Isolation of the Mitochondria

32 T75 dishes are used for each donor. The cells at confluence are washed twice with pH 7.2 PBS buffer (sodium phosphate buffer pH 7.2-0.13 M of NaCl, 3 mM of KCl, 8 mM of Na₂HPO₄ and 1.4 M of KH₂PO₄) and are detached by scraping, and then centrifuged at 1500×g at 4° C. for 5 minutes. The cell pellet is washed with the PBS buffer, recentrifuged and then placed in ice. The pellet is taken up in cold homogenization buffer (0.3 M mannitol, 0.1% BSA, 0.2 mM EDTA, 10 mM HEPES, adjusted to pH 7.4 with KOH, 5 times the pellet volume), homogenized on ice with a 2 ml glass homogenizer. The cell suspension is centrifuged at 1000×g at 4° C. for 10 minutes. The supernatant is recentrifuged at 10 000×g at 4° C. for 15 minutes. The supernatant contains the cytosolic fraction, and the pellet represents the mitochondrial fraction. The mitochondrial fraction is washed twice with the cold homogenization buffer. The protein concentration is measured according to the Bradford method.

2. Protein Assay (Bradford Method)

a—Preparation of the Calibration Range

BSA stock solution: 50 μg/ml (BIORAD; standard protein)

| Protein | BSA | H₂O |
|---|---|---|
| (μg/tube) | (μl) | (μl) |
| 0 | 0 | 800 |
| 1 | 20 | 780 |
| 2 | 40 | 760 |
| 3 | 60 | 740 |
| 4 | 80 | 720 |
| 5 | 100 | 700 |
| 6 | 120 | 680 |
| 8 | 160 | 640 |

200 μl of Coomassie Blue G250 are added to each tube. The blue is prepared extemporaneously by five-fold dilution of the stock solution.

b—Preparation of the Samples
if the protein concentration >3 mg/ml, the cell extracts are diluted 100-fold, and 100 μl of the dilution are then taken
+700 μl of milliQ water
+200 μl of blue
if the protein concentration is <3 mg/ml, 10 μl of cell extract are taken.
+790 μl of milliQ water
+200 μl of blue

c—Sample Assay

The samples are homogenized by vortexing and, after leaving to stand for 5 minutes, the optical density is then read on a spectrophotometer at a wavelength of 595 nm.

3. Assay of the Mitochondrial Aconitase Activity

a—Reagents
25 mM pH 7.5 TRIS buffer (Sigma)
sodium citrate (Sigma)
isocitrate dehydrogenase (Sigma)
MnCl₂ (Sigma)

b—Principle of the Mitochondrial Aconitase Activity Assay

The mitochondrial aconitase activity is quantified by measuring the absorbance at 340 nm in a reaction medium containing 0.2 mM NADP⁺, 5 mM of sodium citrate and one unit/ml of isocitrate dehydrogenase in 25 mM Tris-HCl plus 0.6 mM MnCl₂ and 0.05% Triton X-100.

For the assay, 50 pg of mitochondrial protein are added to 1.0 ml of reaction medium at 25° C. The measurements at 340 nm are recorded in 1 cm cells at 5-minute intervals and the mitochondrial aconitase activity is calculated according to the linear increase in absorbance at 340 nm over about 5 minutes. The activity is obtained by using a molar extinction coefficient for NADPH of 6.22×10³ M⁻¹ cm⁻¹ and by assuming the conversion by the isocitrate dehydrogenase of one molecule of citrate into one molecule of NADPH.

4. Mitochondrial Aconitase Assay by Western Blotting (WB)

a—Assay Principle

The protein electrophoresis is performed in a polyacrylamide minigel of 1 mm to 1.5 mm thickness, under denaturing and reductive conditions, in discontinuous buffer according to the Laemmli method (Nature, 1970; 227, 680). Gels containing 12% T and 2.7% C allow the low molecular weight proteins (20 to 120 kDa) to be separated. Gels containing 8% T and 2.7% C allow the high molecular weight proteins (35 to 250 kDa) to be separated.

The solutions required for producing the gels are presented in Appendix A.

Separating Gel

The gel is poured at least two hours before migration.

Pouring of the gel is performed using a pipette up to about 0.5 mm from the bottom of the comb provided for the concentrating gel. Absolute ethanol is added to the surface to obtain a uniform base line (±1 ml/gel).

Concentrating Gel

The ethanol is removed. 2.5 ml of gel are poured out using a polyethylene Pasteur transfer pipette (Biorad) and the combs are then inserted. The gel is polymerized after one hour.

b—Preparation of the Samples

The mitochondrial proteins are subjected to electophoresis on polyacrylamide gel containing 12% T under reductive Laemmli conditions. The samples (25-40 μg of protein) are reduced for 5 minutes at 100° C. in the deposition buffer. After depositing the samples and the markers, migration is performed at 200V for 1 hour in a 50 mM Tris-HCl, 100 mM glycine, 2 mM EDTA pH 8.4 buffer containing 0.1% SDS.

c—Electrophoresis

Deposits

The samples are heated at 95° C. for 5 minutes.

The volume to be deposited depends on the desired amount of protein (maximum volume=25 μl for a 1 mm gel and 40 μl for a 1.5 mm gel). The reference amount is 10 μg of protein, corresponding to 10 μl; it is then adapted according to the expression of the target protein.

The combs are removed. 200 ml of 1× migration buffer are poured onto the gels, into the central compartment between the two gels, and then into the tank up to the quarter level.

The samples are deposited using a tapered tip fitted onto the micropipette and 10 μl of prestained low molecular weight controls (Biorad, Prestained SDS-PAGE standards Low Range) or high molecular weight controls (Amersham, Full Range Rainbow).

Migration

The electrophoresis is performed at room temperature, at 200V. This electrophoresis is stopped when the migration front has left the gel (about 40 minutes of migration).

Semi-Dry Transfer of the Proteins onto Membrane

Two thick sheets of filter paper (Biorad) and cellulose membranes (Biorad) are soaked in Towbin transfer buffer (PNAS, 1979, 76 (9) 4350-4) (see Appendix B).

In the semi-dry transfer apparatus (Biorad), a sheet of wet thick filter paper is placed on the anode.

Once the migration is complete, the concentrating gel is removed and the separating gel is fitted on the cellulose membrane. The membrane comprising the gel is deposited on the sheet of filter paper. The second sheet of filter paper is deposited on the gel.

During the manufacture of the “sandwich”, care is taken to remove any air bubbles using a glass rod so as not to hamper the transfer. The apparatus is closed with a lid constituting the cathode. The protein transfer is performed at 10V for 90 minutes.

Staining with Ponceau Red

To check the quality of the transfer, the proteins are stained with Ponceau Red (Sigma). The cellulose membrane is rinsed with milliQ water and then soaked once in a bath of Ponceau Red for 10 minutes with stirring. It is then washed in several baths of milliQ water until the coloration remains only on the protein bands. The membrane is placed in a plastic bag and scanned. The protein bands may be quantified to determine the total amount of transferred protein.

Blocking of the Non-Specific Binding Sites

The membrane is stirred overnight at 4° C. or for 90 minutes at room temperature in a solution for blocking the non-specific binding sites, constituted of 5% skimmed milk (Régilait) in PBS-T buffer (cf. Appendix B) (20 ml/membrane).

Immunodetection

The references and the optimum dilutions of the antibodies are given in Appendix C.

After blocking the non-specific sites, the membrane is rapidly rinsed in PBS-T. This membrane is placed in contact with the primary antibody diluted to the optimum concentration in PBS-T with or without 5% milk (m/v) depending on the antibody, for 60 minutes with stirring at room temperature or overnight at 4° C.

It is then rinsed rapidly for three times 10 minutes in PBS-T in order to remove the excess non-bound free antibody. It is then placed in contact with the appropriate secondary antibody coupled to peroxidase, diluted in PBS-T or 5% milk (5 ml) with stirring at room temperature. After incubation for 45 minutes, it is rinsed rapidly twice and then washed 5 times for 5 minutes with PBS-T buffer and a final time in 1× PBS. After draining, it is placed on kitchen cling film (SARAN), protein side upwards.

The membrane is revealed using a highly sensitive chemoluminescence detection kit (Amersham; ECL Western blotting), using luminol as peroxidase substrate. Under the action of peroxidase and an amplifier, the luminol is oxidized and passes into a transient excited state. Return to the ground state takes place by emission of photons, which strike an autoradiography film placed on the membrane.

1 ml of each of the two solutions of the detection kit, i.e. 2 ml, the minimum volume required to cover the membrane, are mixed together. The mixture is immediately poured uniformly onto the membrane and left in contact for exactly one minute at room temperature. The drained membrane is enclosed in Saran cling film and placed in a cassette protected from light, and then covered with a preflashed autoradiography film (Amersham, Hyperfilm ECL). After exposure for 5 minutes, the autoradiography film is revealed. A new film may be exposed if necessary, to optimize the desired signal (up to 1 hour of exposure time). The bands are quantified by means of the Gels Analysts 3.01 software.

5. Mitochondrial Aconitase Assay by IEF

This technique allows separation of the three forms of mitochondrial aconitase, the active form [4Fe-4S]²⁺, the inactive form [3Fe-4S]⁺ and the apoenzyme form, according to their isoelectric point.

50 μg of mitochondrial protein are deposited onto a pH 3-10 IEF gel (Biorad). Migration is performed in the criterion system (Biorad) for 1 hour at 100V; 1 hour at 250V and 30 minutes at 500V. After migration, the gel is transferred onto a nitrocellulose membrane and anti-mitochondrial aconitase Western blotting is performed in accordance with the protocol described in paragraph 4.

Results

The mitochondrial aconitase activity was measured after isolating the mitochondria from these cell cultures. A decrease in mitochondrial aconitase activity with ageing is demonstrated.

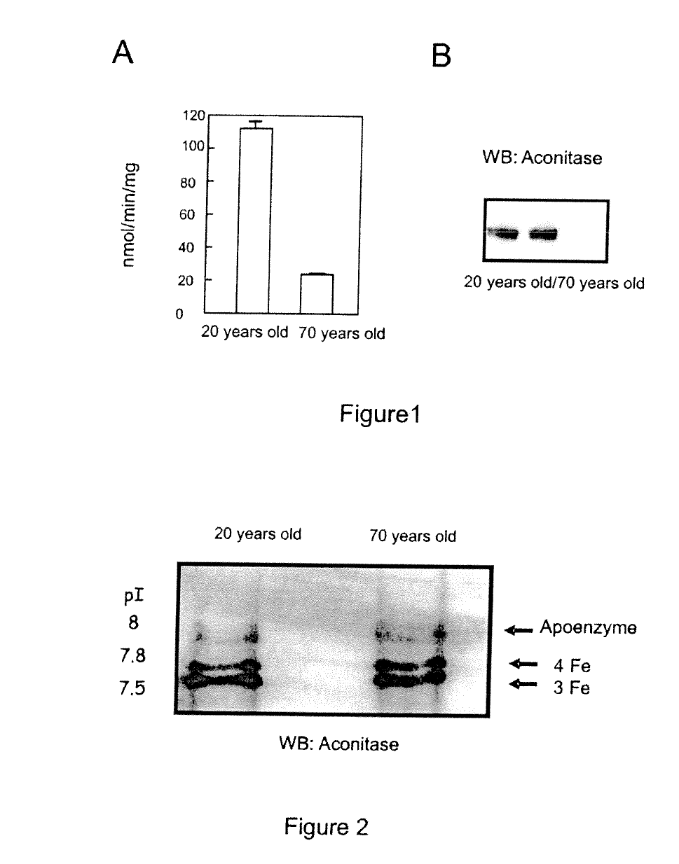

These measurements indicate an approximately 85% decrease in mitochondrial aconitase activity in the mitochondria of the 70-year-old donors in comparison with the 20-year-old donors (cf. FIG. 1).

However, there are no significant differences in expression of the enzyme, i.e. in the amount of enzyme present in the young and old fibroblasts (cf. FIG. 1).

To check whether the decrease in mitochondrial aconitase activity demonstrated in the aged fibroblasts was not due to oxidative damage at the Fe—S centre of the enzyme, the three structural forms of the enzyme (apoenzyme, active form and inactive form) were separated according to their isoelectric point, by isoelectric focusing (IEF). Using this method, a difference with age is demonstrated in the apoenzyme, but there is no difference in the other forms (cf. FIG. 2).

We have shown that the activity of mitochondrial aconitase decreases with age in human dermal fibroblasts in culture. This decrease in activity is not accompanied by a change in expression of the protein with age.

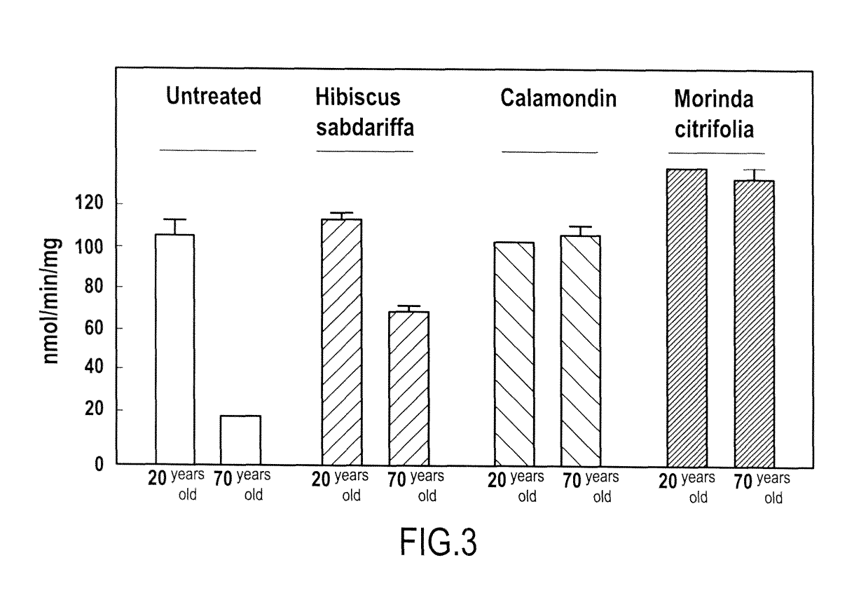

Starting from this result, we characterized the effect of the active agents tested on the mitochondrial aconitase activity in dermal fibroblasts obtained from these donors of different age (cf. FIG. 3 and Table 1).

The results are expressed as a percentage relative to the mitochondrial aconitase activity in untreated fibroblasts from a young donor (20 years old), which constitutes the 100% level.

TABLE 1
Mitochondrial aconitase activity of fibroblasts
from 20- and 70-year-old donors

| (%) | Untreated | Calamondin | M. citrifolia | Hibiscus |
|---|---|---|---|---|
| 20 years old | 100 | 97 | 125 | 115 |
| 70 years old | 18 | 108 | 121 | 67 |

These results indicate that a treatment with these extracts makes it possible to protect the mitochondrial aconitase against the oxidative action. There is no significant activation of mitochondrial aconitase, 48 hours after treating the fibroblasts from 20-year-old donors with these extracts. On the other hand, the same treatment on fibroblasts from 70-year-old donors results in total protection of the enzyme.

Examples of Cosmetic Formulations

### Example 3

### Cosmetic Composition Comprising a Calamondin Extract

The extract obtained in Example 1 is used in the form as obtained, in the cosmetic composition below:

| | |
|---|---|
| Plant extract of Calamondin (Ex. 1) | 1% |
| Surfactant (Arlacel ® 165 VP) | 5% |
| 95% cetyl alcohol | 1% |
| Stearyl alcohol | 1% |
| Beeswax | 1.5% |
| Oil (Parleam ®) | 8.5% |
| Glyceride tricaprate/caprylate | 3% |
| Silicone oil (Dimethicone 100 CS) | 1% |
| Polymer (Keltrol ®) | 0.35% |
| Sodium hydroxide | 0.04% |
| Tetrasodium EDTA powder | 0.1% |
| Preserving agents | 0.5% |
| Water | qs 100 |

The cosmetic composition is prepared in the usual manner, well known to those skilled in the art, by mixing together the various components in one or more steps.

This composition is a night cream that may be applied to the face daily for several weeks to obtain the anti-ageing cosmetic effects indicated previously.

### APPENDIX A

### 1—Buffers and Solutions Used for the Electrophoresis Gels Under Denaturing and Reductive Conditions, in Discontinuous Buffer

Monomer Solution:

acrylamide/bisacrylamide, 30% T, 2.67% C (Biorad)

Resolution gel buffer: Tris-HCl 1.5M pH 8.8

18.15 g of Tris base (Sigma) per 100 ml of distilled water
pH adjusted to 8.8 with 12N HCl

Concentrating gel buffer: 0.5M Tris-HCl pH 6.8

6 g of Tris base per 100 ml of distilled water
pH adjusted to 6.8 with 12N HCl

10× migration buffer: 0.25M Tris pH 8.3, 1.92M glycine; 1% SDS

| | | |
|---|---|---|
| Tris base | 12 | g |
| Glycine (Research Organics Inc) | 57.6 | g |
| 10% SDS (Sigma) | 40 | ml |
| Distilled water | qs 400 | ml |

Ammonium persulfate: (NH₄)₂S₂O₈: (Sigma) at 10% (w/v), i.e. 100 mg/ml

2× Laemmli reductive sample buffer: 0.06M Tris-HCl pH 6.8; 2.3% SDS; 10% glycerol; 0.02% bromophenol blue

| | | |
|---|---|---|
| pH 6.8 0.5M Tris concentrating gel buffer | 6.25 | ml |
| 10% SDS | 11.50 | ml |
| Glycerol | 5 | ml |
| Distilled water | qs 50 | ml |

10× bromophenol blue (saturated solution):

A spatula-tip of bromophenol blue is dispersed in 5 ml of 2× Laemmli buffer. After stirring, sonicating and then centrifuging, the supernatant is recovered.

### II—Electrophoresis Gels

Preparation of the 12% T resolution gel

| | Volumes | | Final |
|---|---|---|---|
| Solutions | for 2 gels (10 ml) | | concentrations |
| Monomer solution | 4.0 | ml | 12% T; 2.7% C |
| Resolution gel buffer | 2.5 | ml | 0.375M |
| 10% SDS | 100 | μl | 0.1% |
| Ammonium persulfate (10%) | 50 | μl | 0.05% |
| TEMED (Research Organics Inc.) | 5 | μl | — |
| Distilled water | 3.4 | ml | — |

Preparation of the 8% T concentrating gel

| | Volumes | | Final |
|---|---|---|---|
| Solutions | for 2 gels (5 ml) | | concentrations |
| Monomer solution | 2 | ml | 8% T; 2.7% C |
| Resolution gel buffer | 1.25 | ml | 0.375M |
| 10% SDS | 50 | μl | 0.1% |
| Ammonium persulfate (10%) | 25 | μl | 0.05% |
| TEMED (Research Organics Inc.) | 5 | μl | — |
| Distilled water | 4.2 | ml | — |

### APPENDIX B

### Solutions for the Transfer and Immunodetection

Towbin Transfer Buffer:

25 mM Tris-HCl, pH 8.3; 192 mM glycine; 20% methanol

| | | |
|---|---|---|
| Tris base | 3.03 | g |
| Glycine (Research Organics Inc.) | 14.4 | g |
| to be dissolved in 100 ml of distilled water | | |
| Methanol | 200 | ml |
| Distilled water | qs 1000 | ml |

PBS-T Buffer

Tenfold dilution of 10×PBS (Invitrogen)

+0.1% Tween 20 (Sigma)

Ponceau Red (Sigma)

0.1% (w/v) solution in 5% acetic acid solution

### APPENDIX C

### List of Primary and Secondary Antibodies

| | | | Incubation |
|---|---|---|---|
| Antibody | Reference | Dilution | time |
| Human | not commercial | 1/1000 | 1 hour |
| anti-aconitase | (cf. ref. below) | | |
| Anti-rabbit | Amersham | 1/50 000 | 1 hour |
| IgG HRP | | | |

Ref.: Bulteau et al. Biochemistry, 2003; 42, 14846-14855

1. A method of cosmetic care for preventing or retarding the appearance of the signs of ageing of the skin or for attenuating the effects thereof, said method being characterized in that it comprises the delivery, to at least part of the skin of the face or body, of an effective amount of at least one cosmetically acceptable active agent that activates or stimulates the activity of cutaneous mitochondrial aconitase.

2. The method of claim 1, wherein said active agent is incorporated into a cosmetic composition also comprising at least one cosmetically acceptable excipient.

3. The method of claim 1, wherein said active agent comprises or is essentially consisting of a plant extract obtained from at least one plant chosen from the group consisting of the genus Citrus, Citrus hybrids obtained from the crossing of plant species, the genus Morinda, and the genus Hibiscus.

4. The method of claim 1, wherein said active agent comprises or is essentially consisting of a plant extract obtained from at least one hybrid belonging to the genus×Citrofortunella, resulting from the crossing of the genera Citrus and Fortunella.

5. The method of claim 1, wherein said active agent is an extract of calamondin fruit (×Citrofortunella microcarpa).

6. The method of claim 1, wherein said active agent comprises or is essentially consisting of a plant extract obtained from at least one plant belonging to the plant species Morinda citnfolia.

7. The method of claim 1, wherein said active agent is an extract of Morinda citnfolia fruit.

8. The method of claim 1, wherein said active agent comprises or is essentially consisting of a plant extract obtained from at least one plant belonging to the plant species Hibiscus sabdariffa.

9. The method of claim 1, wherein said active agent is an extract of Hibiscus sabdariffa blossom.

10. The method of claim 1, wherein the concentration of said active agent is between 0.001% and 5%.

11. The method of claim 1, wherein the concentration of said active agent is from 1% to 3% by weight of the composition.

12. The method of claim 1, wherein said active agent that activates or stimulates the expression of the acotinase activity is associated with a molecule or an extract that inhibits phosphodiesterases.

13. The method of claim 1, wherein said active agent that activates or stimulates the expression of the acotinase activity is associated with caffeine.

14. The method of claim 2, wherein said cosmetic composition is formulated in the form of a serum, a lotion, an emulsion, a cream, a hydrogel, a mask, a stick, or a patch.

15. A method of cosmetic care for damaged skin, wherein said method comprises the delivery, to at least part of the skin of the face or body, of an effective amount of a cosmetically acceptable active agent that activates or stimulates the activity of cutaneous mitochondrial aconitase.

16. The method of claim 15, wherein said active agent is incorporated into a cosmetic composition also comprising at least one cosmetically acceptable excipient.

17. The method of claim 1, wherein said active agent comprises or is essentially consisting of a plant extract obtained from at least one plant chosen from the group consisting of the genus Citrus, Citrus hybrids obtained from the crossing of plant species, the genus Morinda, and the genus Hibiscus.

18. The method of claim 15, wherein said active agent comprises or is essentially consisting of a plant extract obtained from at least one hybrid belonging to the genus×Citrofortunella, resulting from the crossing of the genera Citrus and Fortunella.

19. The method of claim 15, wherein said active agent is an extract of calamondin (×Citrofortunella microcarpa).

20. The method of claim 15, wherein said active agent comprises or is essentially consisting of a plant extract obtained from at least one plant belonging to the plant species Morinda citnfolia.

21. The method of claim 15, wherein said active agent comprises or is essentially consisting of a plant extract obtained from at least one plant belonging to the plant species Hibiscus sabdariffa.

22. The method of claim 15, wherein the concentration of said active agent is between 0.001% and 5%.

23. The method of claim 15, wherein said active agent that activates or stimulates the expression of the acotinase activity is associated with a molecule or an extract that inhibits phosphodiesterases.

24. The method of claim 15, wherein said active agent that activates or stimulates the expression of the acotinase activity is associated with caffeine.

25. The method of claim 16, wherein said cosmetic composition is formulated in the form of a serum, a lotion, an emulsion, a cream, a hydrogel, a mask, a stick, or a patch.
